(19) United States
(12) Patent Application Publication
(10) Pub. No.: US 2023/0240838 A1
(43) Pub. Date: Aug. 3, 2023

(54) Tissue Repair Medical Devices and Methods

(71) Applicants: Cook Medical Technologies LLC, Bloomington, IN (US); Cook Biotech Incorporated, West Lafayette, IN (US)

(72) Inventors: Alycia Berman, West Lafayette, IN (US); Alex Brethauer, Bloomington, IN (US); Christopher A. Carruthers, Winston-Salem, NC (US); Michelle Chutka, West Lafayette, IN (US); Alex de Poix, West Lafayette, IN (US); Shaun Davis Gittard, Winston-Salem, NC (US); Katie Herget, Lafayette, IN (US); Michael Hiles, Lafayette, IN (US); George Malinee, Lafayette, IN (US); Elena Monroe, Libertyville, IL (US); Bhavin B. Shah, West Lafayette, IN (US); Brayden Smith, Lafayette, IN (US)

(73) Assignees: Cook Medical Technologies LLC, Bloomington, IN (US); Cook Biotech Incorporated, West Lafayette, IN (US)

(21) Appl. No.: 18/102,320

(22) Filed: Jan. 27, 2023

Related U.S. Application Data
(60) Provisional application No. 63/304,210, filed on Jan. 28, 2022.

(51) Int. Cl. A61F 2/18

(52) U.S. Cl. CPC A61F 2/18; A61F 2002/183

(57) ABSTRACT

Medical devices for repairing defects in tissue, such as perforations in the tympanic membrane of an animal, methods of manufacturing medical devices, methods of implanting a medical device in a defect in a tissue; and methods of repairing a defect in a tissue are described. In one form, a medical device includes an overlay member having multiple arms and defining an overlay member passageway, an underlay member having multiple flaps and defining an underlay member passageway, and a securement member extending through the overlay member passageway and the underlay member passageway. In methods, the multiple flaps of the underlay member can be sequentially advanced into a defect in a tissue, such as a perforation in a tympanic membrane of an animal.

### FIELD

The disclosure relates generally to the field of medical devices and related methods. More particularly, the disclosure relates to medical devices useful for repairing a defect in tissue, such as a perforation in the tympanic membrane, methods of manufacturing medical devices, methods of implanting a medical device in a defect in a tissue, and methods of repairing a defect in a tissue.

### BACKGROUND

Perforations in tissue can have both temporary and long-term impact on quality of life. For example, a perforation in the tympanic membrane can lead to temporary, recurrent infections, but can also lead to permanent hearing loss. Currently, perforations in the tympanic membrane are repaired with gel packing material that secures a repair graft in place while the patient is under general anesthesia. Unfortunately, current procedures fail to provide an approach for treating tympanic membrane perforations in patients that are not candidates for general anesthesia and fail to provide clinicians with an option for placement and securing graft while treating these perforations without utilizing gel-packing material as a support for a repair graft. While some medical devices have been developed to treat perforations in the tympanic membrane, these medical devices lack sufficient structural support to maintain the medical device in place without use of the gel-packing material.

A need exists, therefore, for new and useful medical devices for repairing defects in tissue, such as a perforation in the tympanic membrane, methods of manufacturing medical devices, methods of implanting a medical device in a defect in a tissue, and methods of repairing a defect in a tissue.

### BRIEF SUMMARY OF SELECTED EXAMPLES

Various example medical devices are described.

An example medical device comprises an overlay member comprising an overlay member body having a first overlay member surface, a second, opposing overlay member surface, a first arm, a second arm, a third arm, and a fourth arm, and defining a first overlay member passageway extending through the overlay member body from the first overlay member surface to the second overlay member surface; an underlay member comprising an underlay member body having a first underlay member surface and a second, opposing underlay member surface, the underlay member body defining a peripheral edge, a first slit extending inwardly from the peripheral edge, a second slit extending inwardly from the peripheral edge, a third slit extending inwardly from the peripheral edge, a fourth slit extending inwardly from the peripheral edge, and a first underlay member passageway extending through the underlay member body from the first underlay member surface to the second underlay member surface; and a securement member extending through the first overlay member passageway and the first underlay member passageway.

Another example medical device comprises an overlay member comprising an overlay member body having a first overlay member surface, a second, opposing overlay member surface, a first arm, a second arm, a third arm, and a fourth arm, and defining a first curvilinear edge disposed between and connecting the first and second arms, a second curvilinear edge disposed between and connecting the second and third arms, a third curvilinear edge disposed between and connecting the third and fourth arms, and a fourth curvilinear edge disposed between and connecting the fourth and first arms, a first overlay member passageway extending through the overlay member body from the first overlay member surface to the second overlay member surface, and a second overlay member passageway extending through the overlay member body from the first overlay member surface to the second overlay member surface; an underlay member comprising an underlay member body having a first underlay member surface and a second, opposing underlay member surface, the underlay member body defining a peripheral edge, a first slit extending inwardly from the peripheral edge, a second slit extending inwardly from the peripheral edge, a third slit extending inwardly from the peripheral edge, a fourth slit extending inwardly from the peripheral edge, a first underlay member passageway extending through the underlay member body from the first underlay member surface to the second underlay member surface, and a second underlay member passageway extending through the underlay member body from the first underlay member surface to the second underlay member surface; and a securement member extending through the first overlay member passageway, the first underlay member passageway, the second underlay member passageway, and the second overlay member passageway.

Another example medical device comprises a cross-shaped overlay member formed of a synthetic material, the overlay member comprising an overlay member body having a first overlay member surface, and a second, opposing overlay member surface, and defining a first overlay member passageway extending through the overlay member body from the first overlay member surface to the second overlay member surface; an underlay member formed of a bioremodelable material, the underlay member comprising an underlay member body having a first underlay member surface and a second, opposing underlay member surface, the underlay member body defining a peripheral edge, a slit extending inwardly from the peripheral edge, and a first underlay member passageway extending through the underlay member body from the first underlay member surface to the second underlay member surface; and a securement member extending through the first overlay member passageway and the first underlay member passageway.

Various example methods of manufacturing a medical device are described.

An example method of manufacturing a medical device comprises forming an overlay member; forming an underlay member; contacting the overlay member with the underlay member; and forming an attachment between the overlay member and the underlay member.

Various example methods of implanting a medical device in a defect in a tissue of an animal, such as a human being, are described.

An example method of implanting a medical device in a defect in a tissue comprises placing a medical device according to an embodiment in a position in which the underlay member is positioned relatively closer to the defect and the overlay member is positioned relatively further from the defect; moving a tool toward the medical device until the distal end of the tool extends beyond an edge of the overlay member of the medical device at a position between adjacent arms of the overlay member and contacts a flap of the underlay member of the medical device; moving the tool to advance a peripheral portion of the flap contacted by the tool until at least the peripheral portion of the flap passes through the defect of the tissue; and retracting the tool away from the medical device until the overlay member of the medical device is positioned between the distal end of the tool and the defect. The sequence of the steps of moving a tool toward the medical device, moving the tool to advance a peripheral portion of the flap, and retracting the tool can be repeated a suitable number of times based on the number of flaps defined by the underlay member of the medical device. An optional step of repositioning the medical device can be included. In particular examples, the defect is a perforation and the tissue is a tympanic membrane of an animal, such as a human being.

Various example methods of repairing a defect in a tissue of an animal, such as a human being, are described.

An example method of repairing a defect in a tissue comprises placing a medical device according to an embodiment in a position in which the underlay member is positioned relatively closer to the defect and the overlay member is positioned relatively further from the defect; moving a tool toward the medical device until the distal end of the tool extends beyond an edge of the overlay member of the medical device at a position between adjacent arms of the overlay member and contacts a flap of the underlay member of the medical device; moving the tool to advance a peripheral portion of the flap contacted by the tool until at least the peripheral portion of the flap passes through the defect of the tissue; retracting the tool away from the medical device until the overlay member of the medical device is positioned between the distal end of the tool and the defect; and allowing a suitable period of time to pass during which the position of the medical device is maintained, such as a period of time suitable for the underlay member to remodel or otherwise become incorporated into tissue of the animal in which the method is being performed. The sequence of the steps of moving a tool toward the medical device, moving the tool to advance a peripheral portion of the flap, and retracting the tool can be repeated a suitable number of times based on the number of flaps defined by the underlay member of the medical device. An optional step of repositioning the medical device can be included. In particular examples, the defect is a perforation and the tissue is a tympanic membrane of an animal, such as a human being.

Additional understanding of the inventive medical devices and methods can be obtained by reviewing the detailed description of selected examples, below, with reference to the appended drawings.

### BRIEF DESCRIPTION OF THE DRAWINGS

FIG. 1 is a top view of an example medical device.

FIG. 1A is a top view of an alternative medical device.

FIG. 2 is a bottom view of the medical device illustrated in FIG. 1.

FIG. 2A is a bottom view of the alternative medical device illustrated in FIG. 1A.

FIG. 3 is a side view of the medical device illustrated in FIG. 1.

FIG. 4 is a top view of the overlay member of the medical device illustrated in FIG. 1.

FIG. 5 is a top view of the underlay member of the medical device illustrated in FIG. 1.

FIG. 6 is a top view of another example medical device.

FIG. 7 is a bottom view of the medical device illustrated in FIG. 6.

FIG. 8 is a side view of the medical device illustrated in FIG. 6.

FIG. 9 is a top view of the securement member of the medical device illustrated in FIG. 6.

FIG. 10 is a top view of another example medical device.

FIG. 11 is a bottom view of the medical device illustrated in FIG. 10.

FIG. 12 is a side view of the medical device illustrated in FIG. 10.

FIG. 13 is a top view of another example medical device.

FIG. 14 is a bottom view of the medical device illustrated in FIG. 13.

FIG. 15 is a side view of the medical device illustrated in FIG. 13.

FIG. 16 is a flowchart illustration of an example method of manufacturing a medical device.

FIG. 17 is a flowchart illustration of an example method of implanting a medical device in a defect in a tissue.

FIG. 18 is a flowchart illustration of an example method of repairing a defect in a tissue.

FIG. 19 is a flowchart illustration of an example method of repairing a perforation in a tympanic membrane of an animal.

FIG. 20 is a flowchart illustration of an example method of implanting a medical device in a perforation in a tympanic membrane of an animal, such as a human being.

FIG. 21 is a top view of another example medical device.

FIG. 22 is a bottom view of the example medical device illustrated in FIG. 21.

### DETAILED DESCRIPTION OF SELECTED EXAMPLES

The following detailed description and the appended drawings describe and illustrate various example medical devices, methods of manufacturing a medical device, methods of implanting a medical device in a defect in a tissue, and methods of repairing a defect in a tissue. The description and illustration of these examples are provided to enable one skilled in the art to make and use an example medical device, practice an example method of manufacturing a medical device, practice an example method of implanting a medical device in a defect in a tissue, and practice an example method of repairing a defect in a tissue in accordance with the invention. These examples are not intended to limit the scope of the claims in any manner.

As used herein, the term “defect,” with respect to a tissue, refers to an unnatural discontinuity in the tissue surface or other portion of the tissue. A defect in a tissue is a discontinuity that is not naturally present in the tissue, and may be the result of damage caused to the tissue by injury or disease. A perforation is an example of a defect in a tissue.

As used herein, the term “diameter” refers to the length of a straight line passing through the center of a body, element, or feature from one surface of the body, element, or feature, to another surface of the body, element, or feature and does not impart any particular structural configuration on the body, element, or feature.

As used herein, the term “perforation,” with respect to a tissue, refers to a defect in a tissue of an animal, such as a human being, that forms an aperture passing through or into the tissue. The term does not require any particular size, shape, or configuration of the aperture and is not limited to any particular tissue.

As used herein, the term “secure,” and grammatically related terms, refers to the fixing of one element to another element in a space. The term, on its own, does not require that the elements be in direct contact with each other, and does not require that the fixing last for a particular duration.

As used herein, the term “securement,” and grammatically related terms, refers to an ability of a described element to secure one element to another element. The term, on its own, does not require any particular size, shape, or configuration.

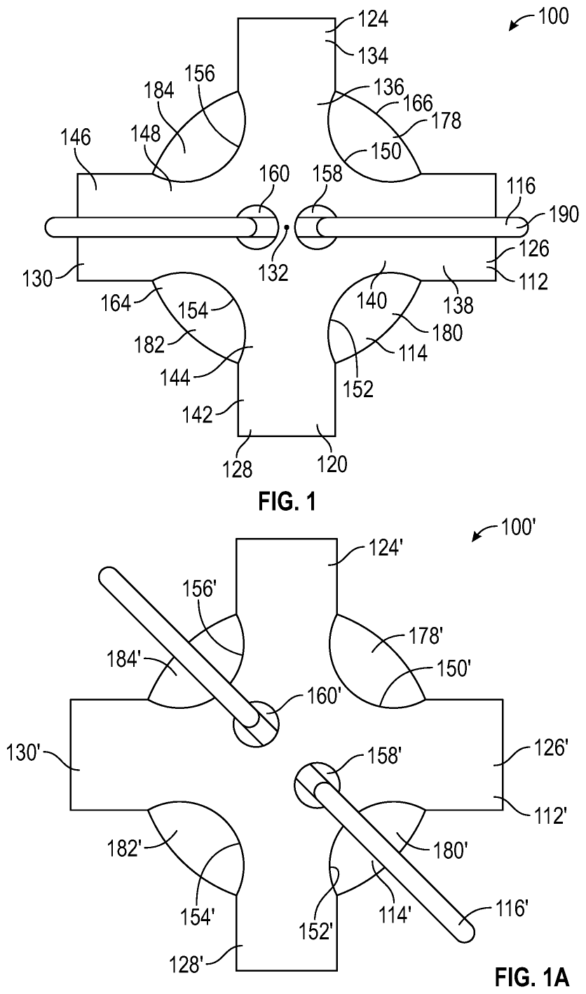

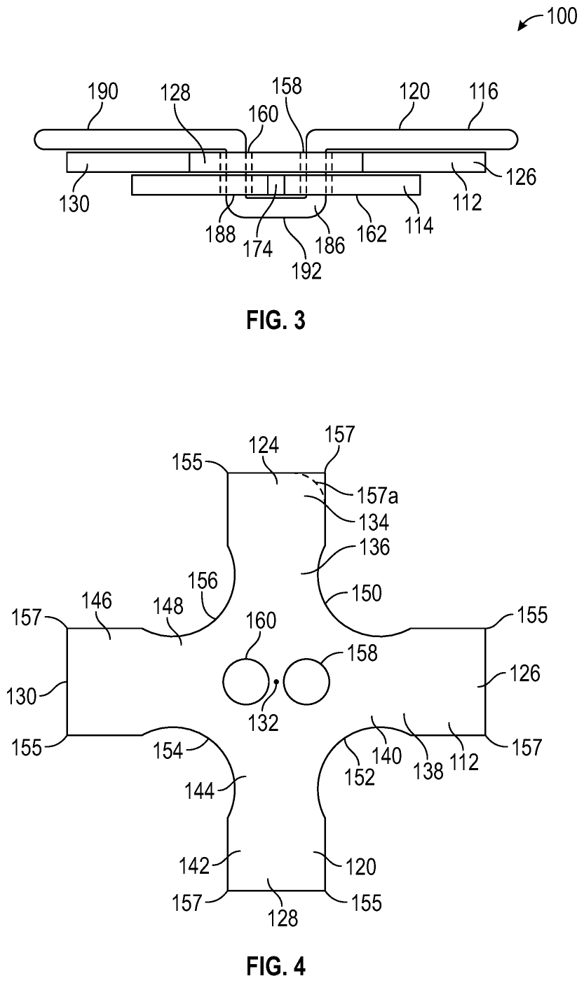

Each of FIGS. 1, 2, 3, 4, and 5 illustrates an example medical device 100 or a component of the medical device 100. The medical device 100 includes an overlay member 112, an underlay member 114, and a securement member 116 that secures the overlay member 112 and the underlay member 114 to each other.

The overlay member 112 is a substantially flat body having a first surface 120 and an opposing second surface 122. The overlay member 112 is generally cross-shaped, having first 124, second 126, third 128, and fourth 130 arms that extend outwardly from a geometric center 132 of the overlay member 112. The first arm 124 extends outwardly in an opposite direction to the third arm 128, and the second arm 126 extends outwardly in an opposite direction to the fourth arm 130. The first arm 124 defines first arm tab 134 and first arm throat 136, the second arm 126 defines second arm tab 138 and second arm throat 140, the third arm 128 defines third arm tab 142 and third arm throat 144, and the fourth arm defines fourth arm tab 146 and fourth arm throat 148. Each of the arms 124, 126, 128, 130 has a length, measured from the geometric center of the overlay member 132 to the terminal end of the respective arm 124, 126, 128, 130, such that the respective arm 124, 126, 128, 130 extends beyond the peripheral edge 166 of the underlay member 114 of the medical device 100 relative to the geometric center 132 of the overlay member 112, as described below. In this example, as best illustrated in FIG. 1, the entirety of each of the arm tabs 134, 138, 142, 146 is positioned beyond the peripheral edge 166 of the underlay member 114 of the medical device 100 relative to the geometric center 132 of the overlay member 112.

The overlay member 112 defines a series of edges 150, 152, 154, 156 that separate pairs of the arms 124, 126, 128, 130 from each other. In this example, as best illustrated in FIGS. 1 and 4, each of the edges 150, 152, 154, 156 is curvilinear and extends inwardly from the tab of one arm to the tab of an adjacent arm. Accordingly, edge 150 is a curvilinear edge that extends inwardly toward the geometric center 132 from the first arm tab 134 to the second arm tab 138. Edge 152 is a curvilinear edge that extends inwardly toward the geometric center 132 from the second arm tab 138 to the third arm tab 142. Edge 154 is a curvilinear edge that extends inwardly toward the geometric center 132 from the third arm tab 142 to the fourth arm tab 146. Edge 156 is a curvilinear edge that extends inwardly toward the geometric center 132 from the fourth arm tab 146 to the first arm tab 134.

The inclusion of curvilinear edges 150, 152, 154, 156 and throats 136, 140, 144, and 148 in the structure of the overlay member 112 provides access to the flaps 178, 180, 182, 184 of the underlay member, as described in more detail below, effectively allowing a clinician to sequentially and individually manipulate the flaps 178, 180, 182, 184 of the underlay member 114 despite the presence of the overlay member 112. While the illustrated embodiments include curvilinear edges, alternative edges can be used in a medical device according to a particular embodiment, including linear edges that connect at a right angle, an acute angle, or other suitable angle, and a combination of curvilinear and linear edges can be used. A skilled artisan will be able to select appropriate edges for inclusion in a medical device according to a particular embodiment based on various considerations, including the nature of the perforation with which the medical device is intended to be used, the extent of access to the underlay member that is anticipated to be needed during placement, and other considerations. Curvilinear edges are considered advantageous at least because of the desirable amount and configuration of the access they provide to the underlay member, but alternative edges, including linear edges as described above, can be used. As illustrated in FIG. 4, each of the arm tabs 134, 138, 142, 146 of the overlay member 112 includes first 155 and second 157 corners. In some embodiments, one or more of the arm tabs 134, 138, 142, 146 includes a corner 155 and a rounded corner 157a, providing an indicator of direction to a clinician during placement of the medical device 100.

While the overlay member 112 in the illustrated example medical device 100 includes four arms 124, 126, 128, 130 and four connecting edges edges 150, 152, 154, 156, a medical device according to a particular embodiment can include an overlay member having any suitable number of arms and a corresponding suitable number of connecting edges. For example, an overlay member can include at least two arms and at least two connecting edges. In this example, the connecting edges can be linear such that the arms are effectively continuous with each other. Curvilinear edges can be used as well, of course. Other numbers of arms and connecting edges considered suitable include, but are not limited to, two arms with two connecting edges, at least two arms with at least two connecting edges, more than two arms with more than two connecting edges, a plurality of arms with a plurality of connecting edges, three arms with three connecting edges, at least four arms with at least four connecting edges, and more than four arms with more than four connecting edges. Also alternatively, an overlay member having one arm and zero connecting edges can be used. A medical device according to this embodiment may be advantageous for use in tissue perforations having limited access or minimal dimensions where elimination of material may provide a beneficial impact on handling properties of the medical device during placement.

Also as best illustrated in FIGS. 1 and 4, the overlay member 112 defines first 158 and second 160 passageways, each of which extends through the entire thickness of the overlay member 112 from the first surface 120 to the second surface 122. As described in detail below, the passageways 158, 160 are configured to receive the securing member 116 in a manner that secures the overlay member 112 to the underlay member 114. The first 158 and second 160 passageways can have any suitable dimensions and can be spaced from each other by any suitable distance. The width of each passageway 158, 160, measured from a point on one edge to an opposite point on an opposing edge, is advantageously at least as large as the width of the securing member 116 used in the medical device 100. A distance of about 1.5 mm between the centers of the first 158 and second 160 passageways is considered suitable. A distance of between about 1 mm and about 2 mm between the centers of the first 158 and second 160 passageways is also considered suitable.

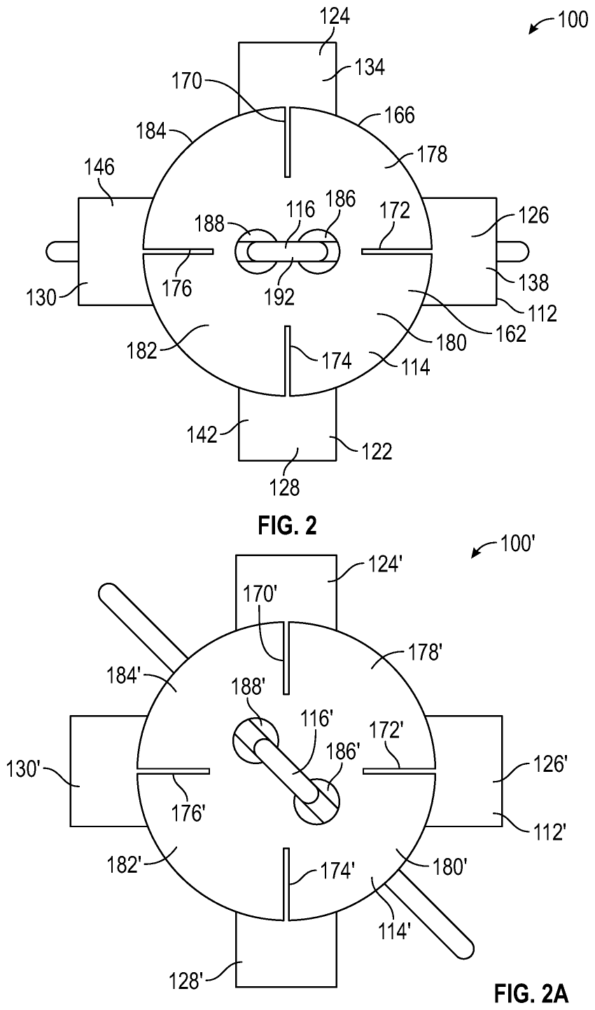

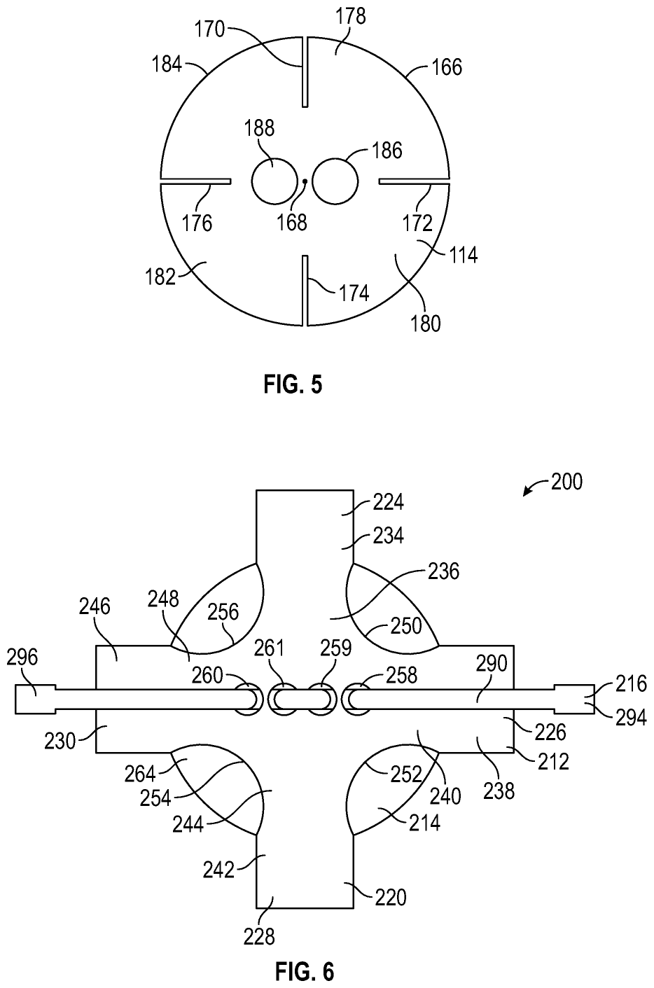

The underlay member 114 is a substantially flat body having a first surface 162 and an opposing second surface 164. The underlay member 114 has a peripheral edge 166 and defines a series of slits that, individually, extend from the peripheral edge 166 toward a geometric center 168. In this example, as best illustrated in FIGS. 2 and 5, the underlay member 114 defines first 170, second 172, third 174, and fourth 176 slits, each of which extends inwardly from the peripheral edge 166 toward the geometric center 168. Each adjacent pair of the slits 170, 172, 174, 176 defines a flap 178, 180, 182, 184 of the material of the underlay member 114 that can be moved above and below the plane of the underlay member 114 without resulting in movement of other flaps 178, 180, 182, 184 of the underlay member 114. The first 170 and second 172 slits define a first flap 178, the second 172 and third 174 slits define a second flap 180, the third 174 and fourth 176 slits define a third flap 182, and the fourth 176 and first 170 slits define a fourth flap 184. This structural configuration is considered particularly advantageous at least because it allows for individual manipulation of portions of the underlay material during placement of a medical device according to an embodiment in, through, or adjacent a perforation in tissue during a repair procedure. Each of the slits can have any suitable length and a skilled artisan will be able to select a suitable length for each slit in a medical device according to a particular embodiment based on various considerations, including the material used in the underlay member, the structure, configuration, and dimensions of any device used in the placement of the medical device in a perforation in body tissue, the nature of the perforation and tissue with which the medical device is intended to be used. Also, all slits in a medical device can have the same length, or slits of different lengths can be used. A slit length that is at least about 25% of the radius of the underlay member is considered advantageous at least because it provides desirable handling characteristics for placing a medical device according to an embodiment in a typical perforation in a tympanic membrane. A slit length that is between about 25% and about 50% of the radius of the underlay member is considered advantageous at least because it provides desirable handling characteristics for placing a medical device according to an embodiment in a typical perforation in a tympanic membrane. A slit length that is about 50% of the radius of the underlay member is considered advantageous at least because it provides desirable handling characteristics for placing a medical device according to an embodiment in a typical perforation in a tympanic membrane. Also advantageously, each of the slits have the same slit length. Also advantageously, each of the slits have a slit length that is about 50% of the radius of the underlay member.

While the underlay member 114 in the illustrated example medical device 100 includes four slits 170, 172, 174, 176 and four flaps 178, 180, 182, 184, a medical device according to a particular embodiment can include an underlay member having any suitable number of slits and a corresponding suitable number of flaps. For example, an underlay member can include at least two slits and at least two flaps. In this example, each of the flaps can be semicircular in shape, but can have non-semicircular shapes as well. Other numbers of slits and flaps considered suitable include, but are not limited to, two slits with two flaps, at least two slits with at least two flaps, more than two slits with more than two flaps, a plurality of slits with a plurality of flaps, three slits with three flaps, at least four slits with at least four flaps, and more than four slits with more than four flaps.

The underlay member 114 in the illustrated embodiment is generally circular in shape, but any shape that provides the desired relative structural arrangement with the overlay member 112 can be used. For example, one advantage of the relative structural arrangement of the overlay member 112 and underlay member 114 of the medical device 100 is that a clinician can extend a tool past a curvilinear edge 150, 152, 154, 156 of the overlay member 112 to contact one of the flaps 178, 180, 182, 184 of the underlay member 114 to position the underlay member 114 in or through a perforation in a tissue being treated. The underlay member in a medical device according to a particular embodiment can have any suitable shape that provides this desired relative structural arrangement between the overlay member and the underlay member of the medical device. Examples of suitable shapes include, but are not limited to, circular, ovoid, and elliptical shapes.

Also as best illustrated in FIGS. 2 and 5, the underlay member 114 defines first 186 and second 188 passageways, each of which extends through the entire thickness of the underlay member 114 from the first surface 162 to the second surface 164. In this example, the first 186 and second 188 passageways are coaxial with the first 158 and second 160 passageways of the overlay member 112. As described in detail below, the passageways 186, 188 are configured to receive the securing member 116 in a manner that secures the overlay member 112 to the underlay member 114. The first 158 and second 160 passageways can have any suitable dimensions and can be spaced from each other by any suitable distance. A distance that is between about 1 mm and about 2 mm between the centers of the first 158 and second 160 passageways is considered suitable. A distance that is between about 1.25 mm and about 1.75 mm between the centers of the first 158 and second 160 passageways is also considered suitable. A distance of about 1.5 mm between the centers of the first 158 and second 160 passageways is also considered suitable.

The securement member 116 is an elongate member having a first surface 190 and an opposing second surface 192. In the fully assembled medical device 100, as best illustrated in FIGS. 1 and 2, the securement member 116 extends through the first 158 and second 160 passageways of the overlay member 112 and the first 186 and second 188 passageways of the underlay member 114 to secure the overlay member 112 and the underlay member 114 to each other. The securement member 116 can form knots or other arrangements if desired for any reason, such as for making the securement between the overlay member 112 and the underlay member 114 more durable. If a knot is used, a knot that provides a minimal profile while also providing a suitable knot strength for maintaining the securement between the overlay member 112 and the underlay member should be selected. While conventional surgical knots are suitable for providing the desired knot strength, they provide a profile that is considered disadvantageously large, and may interfere in the placement of the medical device in a perforation by impacting access of a device used on placement procedures to the flaps of the underlay member. Use of a securement member having a minor width or outer diameter that is equal to or greater than the inner diameter of the smallest of the first 158 and second 160 passageways of the overlay member 112 and the first 186 and second 188 passageways of the underlay member 114 advantageously provides friction that resists removal of the securement member 116, largely eliminating any need for a knot or similar structure. This allows a clinician to easily remove the securement member when and if desired following placement in a tissue.

In this example, the securement member 116 is a substantially flat elongate body. It is noted, though, that the securement member in a medical device according to a particular embodiment can have any suitable structure that permits the securement member to extend through the openings of the overlay and underlay members, as described below, in a manner that secures the overlay and underlay members to each other. Examples of alternative structures suitable for a securement member in a medical device according to a particular embodiment include, but are not limited to, threads, filaments, laminates, flexible rods, and other structures that enable the desired engagement with the overlay member and underlay member, as described above.

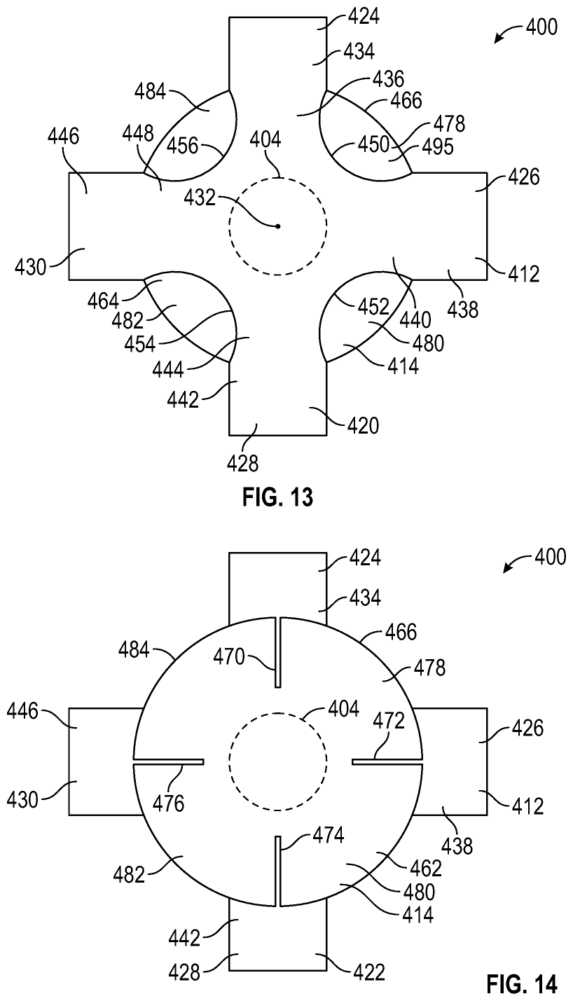

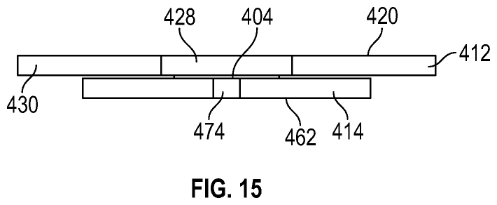

The medical device 100 can include additional components. For example, an adhesive can be applied between the overlay member 112 and the underlay member 114 to reinforce the securement of the overlay member 112 and the underlay member 114 to each other, as described below in connection with the medical device 400 illustrated in FIGS. 13 through 15.

Each of FIGS. 1A and 2A illustrates an alternative medical device 100′ that is identical to medical device 100 except as detailed below. Thus, medical device 100′ includes an overlay member 112′, an underlay member 114′, and a securement member 116′ that secures the overlay member 112′ and the underlay member 114′ to each other. The overlay member 112′ defines multiple arms 124′, 126′, 128′, 130′ arms, each of which extends outwardly from a geometric center (not shown in FIG. 1A) of the overlay member 112′. Each of the arms 124′, 126′, 128′, 130′ defines an arm tab 124′, 126′, 128′, 130′. The overlay member 112′ defines a series of edges 150′, 152′, 154′, 156′, each of which separates a pair of the arms 124′, 126′, 128′, 130′ from each other. The overlay member 112′ defines first 158′ and second 160′ passageways, each of which extends through the entire thickness of the overlay member 112′. The underlay member 114′ has a peripheral edge 166′ and defines multiple slits 170′, 172′, 174′, 176′, each of which extends from the peripheral edge 166′ toward, but not to, a geometric center of the underlay member 114′. The underlay member 114′ defines multiple flaps 178′, 180′, 182′, 184′, each of which extends between circumferentially adjacent slits 170′, 172′, 174′, 176′. The underlay member 114′ defines first 186′ and second 188′ passageways, each of which extends through the entire thickness of the underlay member 114′.

In the example medical device 100 illustrated in FIGS. 1 and 2, the first 158 and second 160 passageways defined by the overlay member 114 are positioned on an axis of the overlay member 112, and of the medical device 100, that extends through the geometric center of the overlay member 112 and arms 126 and 130. As best illustrated in FIG. 1, the passageways 158, 160 are positioned on an axis of the overlay member 112 that bisects opposing arms 126, 130. Also in example medical device 100, the passageways 186, 188 defined by the underlay member 114 are aligned with the passageways 158, 160 defined by the overlay member 112.

In the alternative example medical device 100′ illustrated in FIGS. 1A and 2A, the first 158′ and second 160′ passageways defined by the overlay member 114′ are positioned on an axis of the overlay member 112′, and of the medical device 100′, that extends through the geometric center of the overlay member but does not extend into or through any of the arms 124′, 126′, 128′, 130′ of the overlay member 114′. Indeed, as best illustrated in FIG. 1A, the axis on which the passageways 158′, 160′ are positioned, extends through edge 152′ that separates circumferentially adjacent arms 126′ and 128′ and through edge 156′ that separates circumferentially adjacent arms 124′ and 130′. The passageways 186′, 188′ defined by the underlay member 114′ are aligned with the passageways 158′, 160′ defined by the overlay member 112′. This structural configuration may be advantageous for medical devices intended for use in anatomical locations that provide a caregiver with limited access to the medical device or components of the medical device. For example, this structural configuration is considered advantageous for medical devices intended for use in the repair of perforations or other defects in the tympanic membrane of an animal since the ear canal provides limited access to the medical device during implantation. As best illustrated in FIG. 1A, the securement member 116′ is disposed over relatively small portions of the overlay member 116′ as compared to the securement member 116 in the medical device 100 illustrated in FIG. 1. As compared to the structural configuration in medical device 100, this structural configuration may make it relatively easier to manipulate the securement member 116′ during implantation of the medical device 100′ as part of a method of repairing a perforation in a tympanic membrane of an animal, for example.

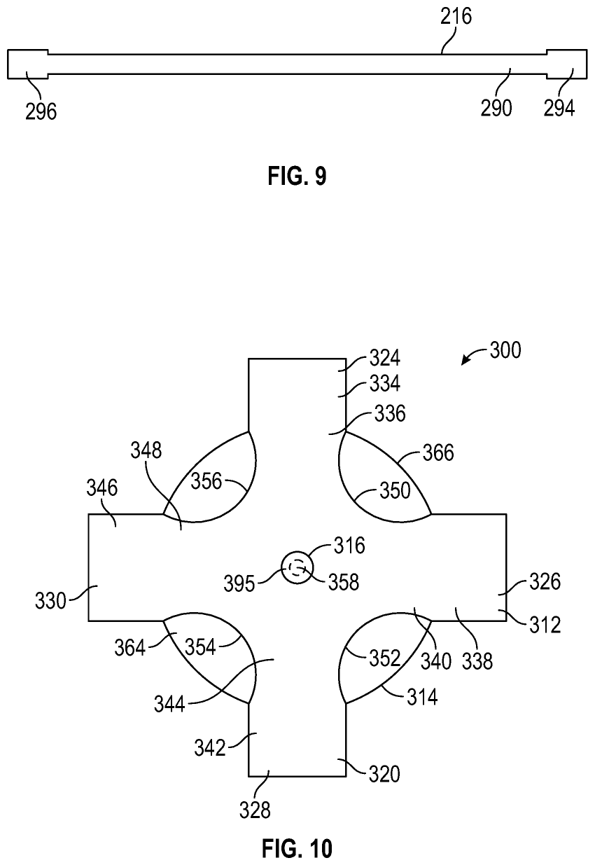

Each of FIGS. 6, 7, 8, and 9 illustrates another example medical device 200 or a component of the medical device 200. The medical device 200 is similar to the medical device 100 illustrated in FIGS. 1 through 3 and described above, except as detailed below. Accordingly, the medical device 200 includes an overlay member 212, an underlay member 214, and a securement member 216 that secures the overlay member 212 and the underlay member 214 to each other.

The overlay member 212 has a first surface 220 and an opposing second surface 222, and first 224, second 226, third 228, and fourth 230 arms that extend outwardly from a geometric center (positioned under the securement member 216 and not visible in the figures) of the overlay member 212. The first arm 224 extends outwardly in an opposite direction to the third arm 228, and the second arm 226 extends outwardly in an opposite direction to the fourth arm 230. The first arm 224 defines first arm tab 234 and first arm throat 236, the second arm 226 defines second arm tab 238 and second arm throat 240, the third arm 228 defines third arm tab 242 and third arm throat 244, and the fourth arm defines fourth arm tab 246 and fourth arm throat 248. The overlay member 212 defines a series of curvilinear edges 250, 252, 254, 256, each of which extends inwardly from the tab of one of the arms 224, 226, 228, 230 to the tab of an adjacent one of the arms 224, 226, 228, 230 to separate the pair of the arms 224, 226, 228, 230 from each other. Each of the arms 224, 226, 228, 230 has a length, measured from the geometric center of the overlay member 232 to the terminal end of the respective arm 224, 226, 228, 230, such that the respective arm 224, 226, 228, 230 extends beyond the peripheral edge 266 of the underlay member 214 of the medical device 200 relative to the geometric center of the overlay member 212, as described below.

In this example, the overlay member 212 defines first 258, second 260, third 259, and fourth 261 passageways, each of which extends through the entire thickness of the overlay member 212 from the first surface 220 to the second surface 222. As described in detail below, the passageways 258, 260, 259, 261 are configured to receive the securing member 216 in a manner that secures the overlay member 212 to the underlay member 214.

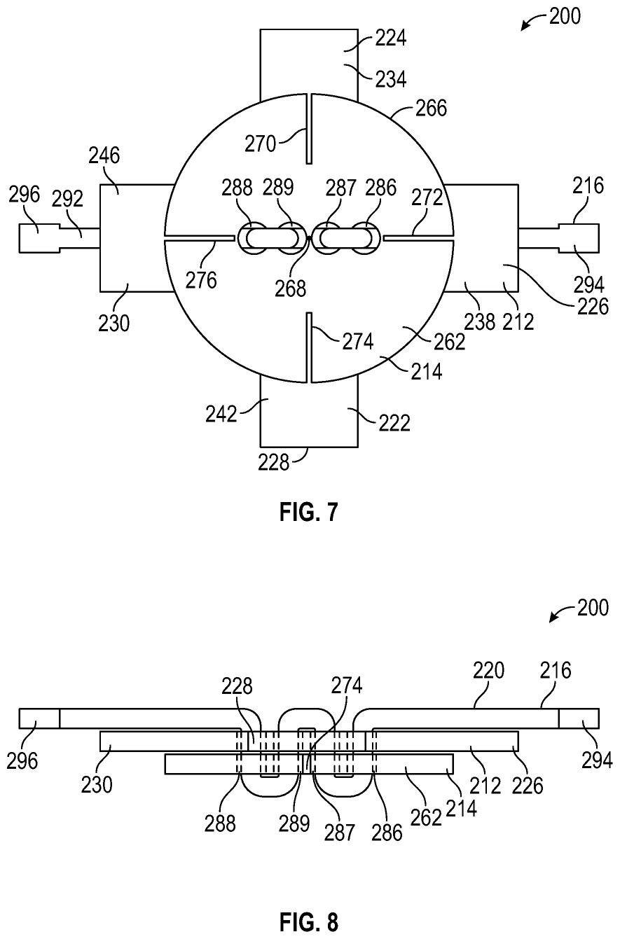

The underlay member 214 has a first surface 262 and an opposing second surface 264. The underlay member 214 has a peripheral edge 266 and defines a series of slits that, individually, extend from the peripheral edge 266 toward a geometric center 268. In this example, as best illustrated in FIG. 7, the underlay member 214 defines first 270, second 272, third 274, and fourth 276 slits, each of which extends inwardly from the peripheral edge 266 toward the geometric center 268. Each adjacent pair of the slits 270, 272, 274, 276 defines a flap 278, 280, 282, 284 of the material of the underlay member 214 that can be moved above and below the plane of the underlay member 214 without resulting in movement of other flaps 278, 280, 282, 284 of the underlay member 214.

In this example, the underlay member 214 defines first 286, second 288, third 287, and fourth 289 passageways, each of which extends through the entire thickness of the underlay member 214 from the first surface 262 to the second surface 264. In this example, the first 286, second 288, third 287, and fourth 289 passageways are coaxial with the first 258, second 260, third 259, and fourth 261 passageways of the overlay member 212. As described in detail below, the passageways 286, 288, 287, 261 are configured to receive the securing member 216 in a manner that secures the overlay member 212 to the underlay member 214.

While the medical device 200 includes four passageways in each of the overlay member 212 and the underlay member, it is noted that any suitable number of passageways can be included in each member in a medical device according to a particular embodiment, and a skilled artisan will be able to select a suitable number of passageways for each member based on a variety of considerations, including the nature of the material used in each of the overlay member, the underlay member, and the securement member. Inclusion of between two and four passageways, inclusively, is each of the overlay member and the underlay member is considered advantageous for a medical device in which at least one of the overlay member and the underlay member is formed of a bioremodelable material, such as small intestine submucosa (SIS), and the securement member is formed of a bioremodelable material, such as SIS.

The securement member 216 has a first surface 290 and an opposing second surface 292 and extends through the first 258, second 260, third 259, and fourth 261 passageways of the overlay member 212 and the first 286, second 288, third 287, and fourth 289 passageways of the underlay member 214 to secure the overlay member 212 and the underlay member 214 to each other. In this example, the securement member 216 defines first 294 and second 296 tabs at respective ends of the securement member 216. Each of the tabs 294, 296 has a width, measured orthogonally to the lengthwise axis of the securement member 216, that is greater than a width measured orthogonally to the lengthwise axis of the securement member 216 at the geometric center of the securement member 216.

Each of FIGS. 10, 11, and 12 illustrates another example medical device 300. The medical device 300 is similar to the medical device 100 illustrated in FIGS. 1 through 3 and described above, except as detailed below. Accordingly, the medical device 300 includes an overlay member 312, an underlay member 314, and a securement member 316 that secures the overlay member 312 and the underlay member 314 to each other.

The overlay member 312 has a first surface 320 and an opposing second surface 322, and first 324, second 326, third 328, and fourth 330 arms that extend outwardly from a geometric center (disposed within passageway 358 and not visible in the figures). The first arm 324 extends outwardly in an opposite direction to the third arm 328, and the second arm 326 extends outwardly in an opposite direction to the fourth arm 330. The first arm 324 defines first arm tab 334 and first arm throat 336, the second arm 326 defines second arm tab 338 and second arm throat 340, the third arm 328 defines third arm tab 342 and third arm throat 344, and the fourth arm defines fourth arm tab 346 and fourth arm throat 348. The overlay member 312 defines a series of curvilinear edges 350, 352, 354, 356, each of which extends inwardly from the tab of one of the arms 324, 326, 328, 330 to the tab of an adjacent one of the arms 324, 326, 328, 330 to separate the pair of the arms 324, 326, 328, 330 from each other.

In this example, the overlay member 312 defines only a single passageway 358 that extends through the entire thickness of the overlay member 312 from the first surface 320 to the second surface 322. As described in detail below, the passageway 358 is configured to receive the securing member 316 in a manner that secures the overlay member 312 to the underlay member 314.

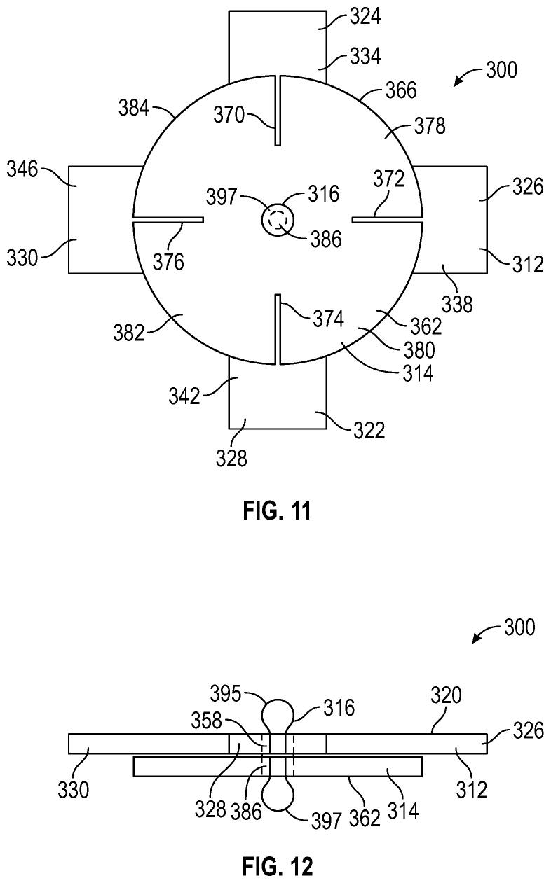

The underlay member 314 has a first surface 362 and an opposing second surface 364. The underlay member 314 has a peripheral edge 366 and defines a series of slits that, individually, extend from the peripheral edge 366 toward a geometric center 368. In this example, as best illustrated in FIG. 11, the underlay member 314 defines first 370, second 372, third 374, and fourth 376 slits, each of which extends inwardly from the peripheral edge 366 toward the geometric center 368. Each adjacent pair of the slits 370, 372, 374, 376 defines a flap 378, 380, 382, 384 of the material of the underlay member 314 that can be moved above and below the plane of the underlay member 314 without resulting in movement of other flaps 378, 380, 382, 384 of the underlay member 314.

In this example, the underlay member 314 defines only a single passageway 386 that extends through the entire thickness of the underlay member 314 from the first surface 362 to the second surface 364. In this example, the passageway 386 is coaxial with the passageway 258 of the overlay member 312. As described in detail below, the passageway 386 is configured to receive the securing member 316 in a manner that secures the overlay member 312 to the underlay member 314.

In this example, as best illustrated in FIG. 12, the securement member 316 is an elongate member having first 395 and second 397 ends, each of which has a bulb-shaped form with a diameter that is greater than a diameter of the securement member 316 measured orthogonally to its lengthwise axis through its geometric center. As such, the securement member 316 is a columnar member having bulb-shaped ends. The securement member 316 extends through the passageway 358 of the overlay member 312 and through the passageway 386 of the underlay member 314, securing the overlay member 312 and the underlay member to each other. Each of the first 395 and second 397 ends has a diameter that is greater than the inner diameter of each of the passageway 358 of the overlay member 312 and through the passageway 386 of the underlay member 314. Advantageously, as best illustrated in FIG. 12, a portion of the first end 395 is in contact with the first surface 320 of the overly member, and a portion of the second end 397 is in contact with the first surface 362 of the underlay member 314. As such, the securement member provides a rivet-like connection between the overlay member 312 and the underlay member 314.

Each of FIGS. 13, 14, and 15 illustrates another example medical device 400. The medical device 400 is similar to the medical device 100 illustrated in FIGS. 1 through 3 and described above, except as detailed below. Accordingly, the medical device 400 includes an overlay member 412 and an underlay member 414. In this example, the medical device does not include a securement member. Rather, as described in more detail below, the overlay member 412 and the underlay member 414 are secured to each other by a contact interface 404 formed between the overlay member 412 and the underlay member 414.

The overlay member 412 has a first surface 420 and an opposing second surface 422, and first 424, second 426, third 428, and fourth 430 arms that extend outwardly from a geometric center 432 of the overlay member 412. The first arm 424 extends outwardly in an opposite direction to the third arm 428, and the second arm 426 extends outwardly in an opposite direction to the fourth arm 430. The first arm 424 defines first arm tab 434 and first arm throat 436, the second arm 426 defines second arm tab 438 and second arm throat 440, the third arm 428 defines third arm tab 442 and third arm throat 444, and the fourth arm defines fourth arm tab 446 and fourth arm throat 448. The overlay member 412 defines a series of curvilinear edges 450, 452, 454, 456, each of which extends inwardly from the tab of one of the arms 424, 426, 428, 430 to the tab of an adjacent one of the arms 424, 426, 428, 430 to separate the pair of the arms 424, 426, 428, 430 from each other. Each of the arms 424, 426, 428, 430 has a length, measured from the geometric center of the overlay member 432 to the terminal end of the respective arm 424, 426, 428, 430, such that the respective arm 424, 426, 428, 430 extends beyond the peripheral edge 466 of the underlay member 414 of the medical device 400 relative to the geometric center 432 of the overlay member 412, as described below.

The underlay member 414 has a first surface 462 and an opposing second surface 464. The underlay member 414 has a peripheral edge 466 and defines a series of slits that, individually, extend from the peripheral edge 466 toward a geometric center 468. In this example, as best illustrated in FIG. 14, the underlay member 414 defines first 470, second 472, third 474, and fourth 476 slits, each of which extends inwardly from the peripheral edge 466 toward the geometric center 468. Each adjacent pair of the slits 470, 472, 474, 476 defines a flap 478, 480, 482, 484 of the material of the underlay member 414 that can be moved above and below the plane of the underlay member 414 without resulting in movement of other flaps 478, 480, 482, 484 of the underlay member 414.

In this example, contact interface 404 is formed between the overlay member 412 and the underlay member 414 and secures the overlay member 412 and the underlay member to each other. The contact interface 404 is an attachment between the overlay member 412 and the underlay member 414 that provides the desired securement between the overlay member 412 and the underlay member. The contact interface 404 can have any suitable size relative to the overlay member 412 and the underlay member 414, but should be large enough to provide the desired securement between the overlay member 412 and the underlay member 414 while being small enough to not extend into any of the slits 470, 472, 474, 476. As best illustrated in FIGS. 13 and 14, the contact interface 404 in this example is substantially circular in shape, having a diameter that is less than the distance between opposing pairs of the slits 470, 472, 474, 476. Also in this example, the contact interface is formed by an adhesive disposed between the overlay member 412 and the underlay member 414.

Any suitable attachment can be used and a skilled artisan will be able to select a suitable attachment for the contact interface between the overlay member and the underlay member in a medical device according to a particular embodiment based on various considerations, including the nature of the material used in each of the overlay member and the underlay member in the medical device. Examples of suitable attachments include, but are not limited to, lamination joints, such as a joint formed between the overlay member and the underlay member by vacuum-pressing the overlay member and the underlay member together, dehydrothermal bonding or crosslinking, adhesives, and other means for attaching one member to another member. Various types of adhesives are considered suitable. For example, dermal wound adhesives, such as Dermabond topical skin adhesive from Ethicon, and silicone rubber adhesives are considered suitable. Dermal wound adhesives are considered advantageous at least because of their well-characterized nature, the relatively short timeframe over which they break down and slough off after application, and the bond strength they provide. These adhesives are particularly advantageous in medical devices that include an overlay member formed of a synthetic material, such as a polyethylene closed cell foam or silicone, and an underlay material formed of a natural material, such as an extracellular matrix (ECM) material or other bioremodelable material. Non-penetrating silicone rubber adhesives are also considered advantageous in these particular medical devices as the silicone-rubber adhesive is not likely to become incorporated into newly formed tissue as the underlay member remodels, and is likely to become less adherent over time, providing for a release of the synthetic overlay member over time. An attachment for the contact interface between the overlay member and the underlay member can also be formed using lyophilization with direct pressure applied to one or both of the overlay member and the underlay member. In these examples, an adhesive can be disposed between the overlay member and the underlay member, but is considered optional. No matter the components selected to form, and the nature of, the attachment for the contact interface between the overlay member and the underlay member in a medical device according to a particular embodiment, the inventors have determined that an attachment having a joint strength that is at least about 0.0103 N is considered advantageous at least because it provides desirable handling characteristics for a medical device during anticipated placement procedures. An attachment having a joint strength that is at least about 0.118 N is also considered advantageous for the same reason, and is considered particularly suitable for medical devices in which the attachment between the overlay member and the underlay member is formed by vacuum pressing the members together.

All components of medical devices according to particular embodiments can be formed of any suitable material. A skilled artisan can select a suitable material for each component in a medical device according to a particular embodiment based on various considerations, including the type of securement formed between the overlay member and the underlay member in the medical device, any desired attachment or other contact interface between the medical device and the particular body tissue with which the medical device is intended to be used, a shape of a perforation defined by the body tissue, and other considerations. Examples of suitable materials for all components of medical devices according to embodiments include, but are not limited to, natural materials, synthetic materials, and combinations of natural and synthetic materials. Allogenic and xenogeneic materials are considered suitable materials for all components. For medical devices intended to be used for repairing a perforation in a body tissue, such as a perforation in a tympanic membrane, ECM materials, such as SIS, and other bioremodelable materials, such as bovine pericardium, are considered particularly advantageous for use in all components of a medical device at least because of the ability of these materials to remodel and integrate into the body tissue. Other examples of suitable ECM materials for use in components of medical devices according to particular embodiments include, but are not limited to, dermis, stomach submucosa, liver basement membrane, urinary bladder submucosa, tissue mucosa, and dura mater. Other examples of suitable natural materials include renal capsule matrix, abdominal fascia, parenchyma, such as abdominal parenchyma, connective tissue, pulmonary or lung ligament, tissue laminates, collagen-containing materials, and natural valve leaflets with or without adjacent vessel wall. Pleura is also considered a suitable natural material, including visceral pleura. Examples of suitable synthetic materials include polymeric materials, such as expanded polytetrafluoroethylene, polyurethane, polyurethane urea, polycarbonate, and polyesters. Absorbable polymers are also considered to be suitable materials, particularly for the underlay member in a medical device according to an embodiment. Examples of suitable absorbable polymers include, but are not limited to, poly lactide, PLGA, PGA, PDO, PCL, poly hydroxybutyrate, and poly ester urea. A skilled artisan will be able to select a suitable material for each component of a medical device according to a particular embodiment based on various considerations. It is considered advantageous for the underlay member to be formed of a material that aids in remodeling of the tissue perforation for which the medical device is being used. The overlay member provides support for the underlay member, access to the underlay member, and anchoring for the medical device once placed in a perforation in tissue. Accordingly, use of a material that aids in remodeling of the tissue is not considered critical for the overlay member, and any biocompatible material that can provide the desired structure and function, as described herein, can be used.

In some embodiments, all components in a medical device are formed of the same material. For example, a medical device according to an embodiment can include a underlay member formed of a bioremodelable material, such as SIS, an overlay member formed of the same bioremodelable material, and a securement member, if included, formed of the same bioremodelable material. Alternatively, in other embodiments, components of a medical device are formed of different materials. For example, a medical device according to an embodiment can include an underlay member formed of a bioremodelable material, such as SIS, an overlay member formed of a synthetic material, such as a polymeric material, and a securement member, if included, formed of the same or different bioremodelable material, the same or different synthetic material, or a material that is different from both the bioremodelable material and from the synthetic material. A medical device according to one particular embodiment includes an underlay material formed of a bioremodelable material, such as SIS, and an overlay member formed of a synthetic material, such as a closed cell polyethylene foam, a silicone, or a biocompatible plastic. If included, a securement member in the medical device according to this particular embodiment can be formed of a bioremodelable material, such as SIS, or a synthetic material, such as nylon. Conventional medical sutures can be used as a securement member in a medical device according to an embodiment, if desired. Other suitable materials for the securement member include, but are not limited to, catgut, SIS sutures, such as SIS spun thread sutures, ECMs, bioremodelable materials, collagen-containing materials, and monofilament. A medical device according to another particular embodiment includes an underlay material formed of a bioremodelable material, such as SIS, and an overlay member formed of a synthetic material, such as a closed cell polyethylene foam or a silicone. An adhesive, such a dermal adhesive or a silicone-rubber adhesive, is used to form a contact interface between the overlay member and the underlay member.

While the overlay member and underlay member are described and illustrated as separate members in the example medical devices described an illustrated herein, a medical device can include a single main body that includes an overlay member and an underlay member integrally formed with each other as a single piece. In these embodiments, the securement member is not necessary to provide an attachment between the overlay member and the underlay member. Medical devices according to these embodiments are advantageously formed of bioremodelable materials or bioresorbable materials, such as bioresorbable polymers like polylactic acid (PLA), PLGA, PGA, PCL, P4HB, or co-polymers, so that the underlay member portion of the medical device provides the desirable aiding of the remodeling process for the tissue lesion while the overlay member portion of the medical device includes the desirable structural properties for providing access to the underlay member during positioning of the medical device and anchoring of the medical device once positioned.

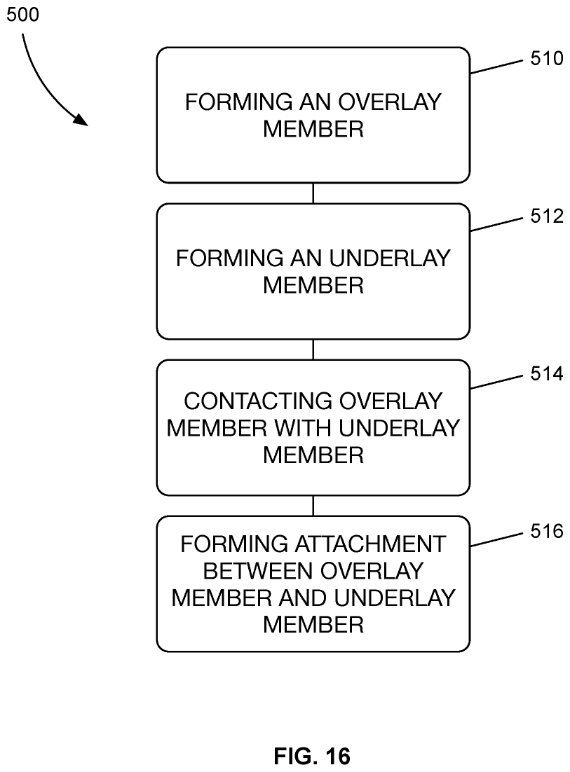

FIG. 16 is a flowchart illustration of an example method 500 of manufacturing a medical device. The method 500 can be performed to manufacture a medical device according to an embodiment of the invention, such as the example medical devices described herein.

An initial step 510 comprises forming an overlay member. This step can be performed using any suitable technique and the overlay member formed can have any suitable size, shape, and structural configuration in accordance with the inventive medical devices, including the example medical devices described herein. A skilled artisan will be able to select a suitable technique for performing the step of forming an overlay member in a particular method of manufacturing a medical device based on various considerations, including the nature of the material used in the forming of an overlay member. Examples of suitable techniques include die cutting, laser cutting, injection molding, 3D printing, and other techniques suitable for forming members of the types of materials described herein for the overlay member.

Another step 512 comprises forming an underlay member. This step can be performed using any suitable technique and the underlay member formed can have any suitable size, shape, and structural configuration in accordance with the inventive medical devices, including the example medical devices described herein. A skilled artisan will be able to select a suitable technique for performing the step of forming an underlay member in a particular method of manufacturing a medical device based on various considerations, including the nature of the material used in the forming of an underlay member. Examples of suitable techniques include die cutting, laser cutting, injection molding, 3D printing, and other techniques suitable for forming members of the types of materials described herein for the underlay member.

Another step 514 comprises contacting the overlay member formed in step 510 with the underlay member formed in step 512. This step can be performed by simply contacting the overlay member and the underlay member, such as by placing the overlay member on top of the underlay member.

Another step 516 comprises forming an attachment between the overlay member and the underlay member. This step can be performed using any suitable technique and a skilled artisan will be able to select a suitable technique for a method of manufacturing a medical device according to a particular embodiment based on various considerations, including the structural features of the overlay member and the underlay member formed in steps 510 and 512. For example, if the overlay member and the underlay members include passageways, this step can comprise passing a securement member through the passageways of the overlay member and the underlay member and forming a knot in the securement member. Other examples techniques considered suitable for this step 516 include, but are not limited to, vacuum pressing the overlay member and the underlay member together, air drying and lyophilizing the overlay member and the underlay member, with or without pressure applied to the overlay member and/or the underlay member, disposing an adhesive between the overlay member and the underlay member, or any combination of these techniques. This step 516 can be performed simultaneously with performance of step 514, or subsequent to completion of step 514.

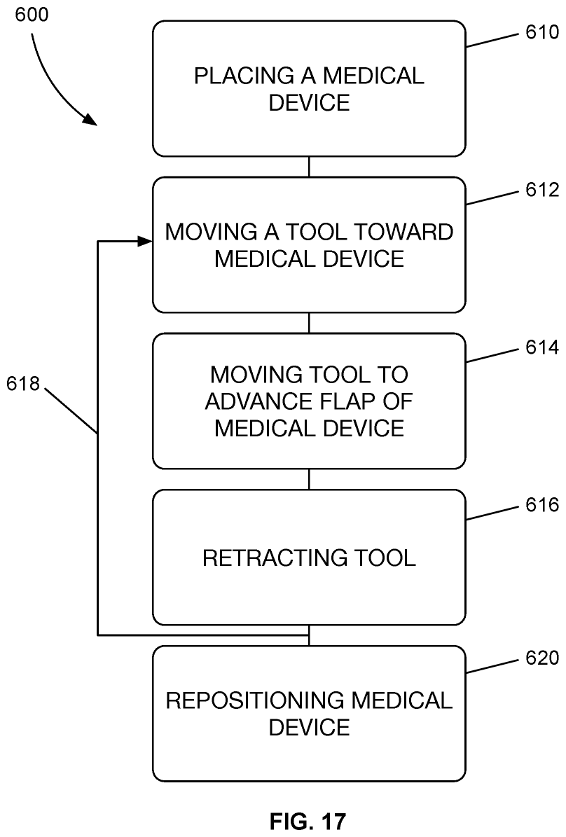

FIG. 17 is a flowchart illustration of an example method 600 of implanting a medical device in a defect in a tissue. The method 600 can be performed to implant a medical device according to an embodiment of the invention, such as the example medical devices described herein. Furthermore, the method 600 can be performed to implant a medical device according to an embodiment at any suitable location in an animal, such as a human being. The method 600 is particularly well suited for implanting medical devices at a point of treatment at which local tissue of the animal has a defect, such as a perforation. For example, the method 600 can be performed to implant a medical device according to an embodiment in a perforated tympanic membrane of an animal, such as a human being. As such, the method 600, and its use of medical devices according to embodiments of the invention, are particularly well suited for repairing perforations in animal tissue, such as the tympanic membrane.

An initial step 610 comprises placing a medical device according to an embodiment in a position in which the underlay member is positioned relatively closer to the defect and the overlay member is positioned relatively further from the defect. This step 610 can be performed by simple handling of the medical device, such as by holding the medical device with forceps or another tool such that the underlay member is positioned relatively closer to the defect and the overlay member is positioned relatively further from the defect. In an example in which the defect is a perforation in the tympanic membrane of an animal, this step can comprise advancing the medical device into the ear canal with the underlay member facing inward relative to the ear canal and the overlay member facing outward with respect to the ear canal. Advantageously, this step is performed until the underlay member is contacting the tissue in which the defect is formed.

Another step 612 comprises moving a tool, such as a tool defining an atraumatic tip, such as a ball or other rounded structure, on its distal end toward the medical device until the distal end of the tool extends beyond an edge of the overlay member of the medical device at a position between adjacent arms of the overlay member and contacts a flap of the underlay member of the medical device.

Another step 614 comprises moving the tool used in step 612 to advance a peripheral portion of the flap contacted by the tool in step 612 until at least the peripheral portion of the flap passes through the defect of the tissue.

Another step 616 comprises retracting the tool used in steps 612 and 614 away from the medical device until the overlay member of the medical device is positioned between the distal end of the tool and the perforation.

After step 616 is completed, the sequence of steps 612, 614, and 616 can be repeated 618 a suitable number of times based on the number of flaps defined by the underlay member of the medical device. For example, for the medical device 100 illustrated in FIGS. 1, 2, and 3, the sequence of steps 612, 614, and 616 can be repeated three times, following the initial performance of these steps, so that at least a peripheral portion of each of the four flaps 178, 180, 182, 184 defined by the underlay member 114 of the medical device 100 has passed through the defect.

An optional step 620 comprises repositioning the medical device if repositioning is considered desirable or necessary. This step can be completed such that either all flaps of the underlay member remain in their respective positions following completion of all iterations of steps 612, 614, and 616, or such that one or more flaps of the underlay member is moved in a manner that brings the one or more flaps of the underlay member back through the defect. In the latter option, steps 612, 614, and 616 can be repeated for the one or more flaps of the underlay member that have been passed back through the defect to position at least a peripheral portion of that particular flap back through the defect. The repositioning can be done using any suitable technique, including by grasping a knot or other portion of the securement member of the medical device, or an arm of the overlay member of the medical device, and applying force so that the medical device rotates, moves laterally, or is otherwise repositioned relative to the defect.

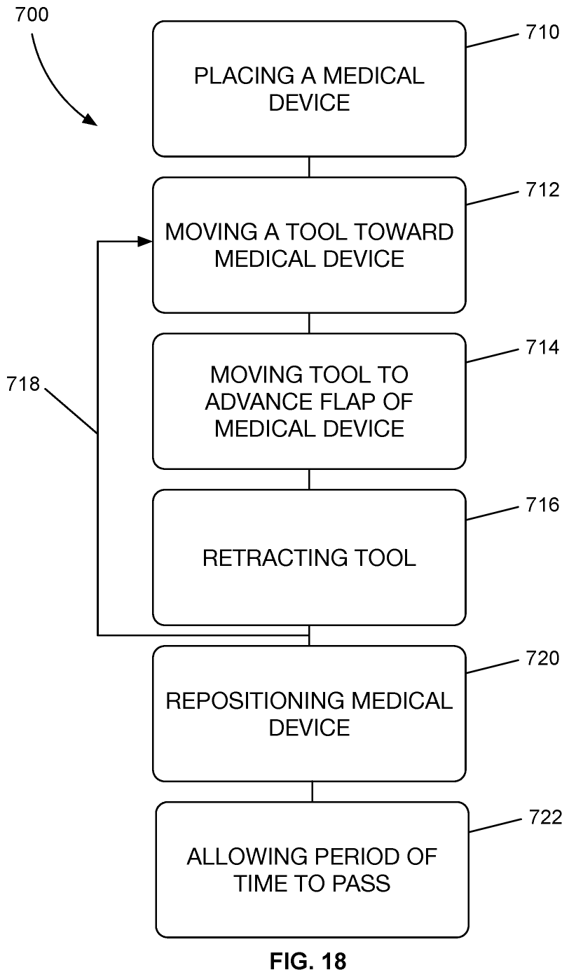

FIG. 18 is a flowchart illustration of an example method 700 of repairing a defect in a tissue. The method 700 includes the method 600, with an additional step. Thus, an initial step 710 comprises placing a medical device according to an embodiment in a position in which the underlay member is positioned relatively closer to the defect and the overlay member is positioned relatively further from the defect. Another step 712 comprises moving a tool, such as a tool defining an atraumatic tip, such as a ball or other rounded structure, on its distal end toward the medical device until the distal end of the tool extends beyond an edge of the overlay member of the medical device at a position between adjacent arms of the overlay member and contacts a flap of the underlay member of the medical device. Another step 714 comprises moving the tool used in step 712 to advance a peripheral portion of the flap contacted by the tool in step 712 until at least the peripheral portion of the flap passes through the defect of the tissue. Another step 716 comprises retracting the tool used in steps 712 and 714 away from the medical device until the overlay member of the medical device is positioned between the distal end of the tool and the defect. After step 716 is completed, the sequence of steps 712, 714, and 716 can be repeated 718 a suitable number of times based on the number of flaps defined by the underlay member of the medical device. An optional step 720 comprises repositioning the medical device if repositioning is considered desirable or necessary.

After the medical device is implanted in this manner, another step 722 comprises allowing a suitable period of time to pass during which the position of the medical device is maintained. The length of the period of time will depend on the nature of the material of the medical device, and of the underlay member of the medical device in particular. For example, in methods in which the underlay member comprises a remodelable material, such as an ECM, a period of time suitable for the underlay member to remodel or otherwise become incorporated into tissue of the animal in which the method is being performed is suitable and considered advantageous. In these methods, the period of time is of a sufficient length to allow the underlay member to complete its remodeling process such that, after allowing the period of time to pass, the perforation will no longer exist and the tissue is repaired. At this point, the overlay member will generally slough off and typically does not require further handling.

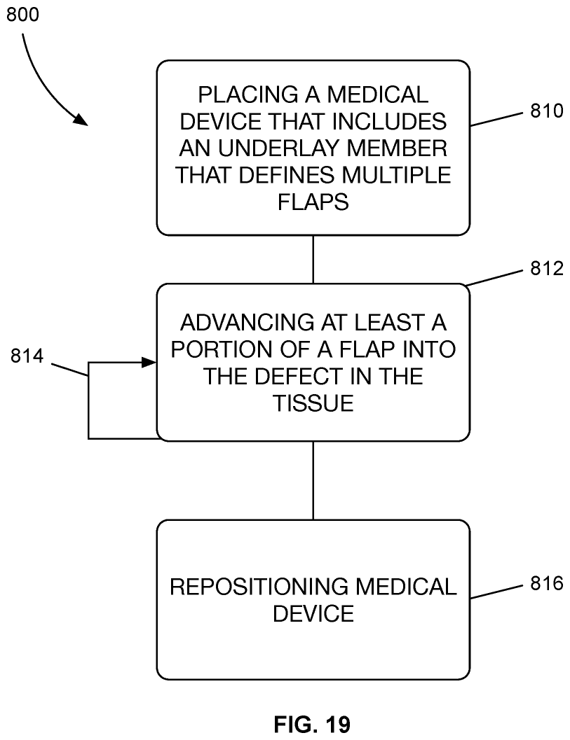

FIG. 19 is a flowchart illustration of another example method 800 of repairing a defect in a tissue. An initial step 810 comprises placing a medical device in a position relative to the defect in which the underlay member is positioned closer to the defect and the overlay member is positioned further from the defect. The medical device includes an underlay member that defines multiple flaps, each of which is circumferentially spaced from at least one other flap of the multiple flaps, such as by a slit. Another step 812 comprises advancing at least a portion, such as a peripheral portion, of a flap of the underlay member into the defect in the tissue. An optional step 814 comprises repeating step 812 for each additional flap defined by the underlay member of the medical device. An optional step 816 comprises repositioning the medical device relative to the defect if repositioning is considered desirable or necessary. Repositioning may include moving the medical device, such as by advancing or retracting a portion of the medical device, such as a flap of the underlay member, relative to the defect in the tissue, rotating the medical device with respect to the defect in the tissue with our without advancing or retracting a portion of the medical device, such as a flap of the underlay member, relative to the defect in the tissue, or other movement of the medical device relative to the defect in the tissue.

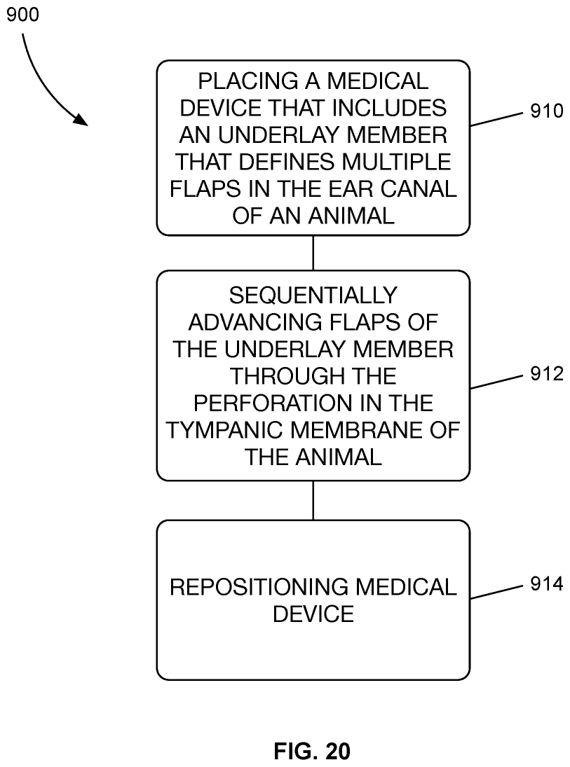

FIG. 20 is a flowchart illustration of an example method 900 of implanting a medical device in a perforation in a tympanic membrane of an animal, such as a human being. An initial step 910 comprises placing a medical device according to an embodiment in a position in an ear canal containing the tympanic membrane of the animal such that the underlay member of the medical device is positioned closer to the perforation and the overlay member is positioned further from the perforation. The medical device includes an underlay member that defines multiple flaps, each of which is circumferentially spaced from at least one other flap of the multiple flaps, such as by a slit. Another step 912 comprises sequentially advancing at least a portion, such as a peripheral portion, of at least two flaps of the underlay member into the perforation in the tympanic membrane. Advantageously, this step 912 comprises sequentially advancing at least a portion, such as a peripheral portion, of all flaps of the underlay member into the perforation in the tympanic membrane. An optional step 914 comprises repositioning the medical device relative to the perforation if repositioning is considered desirable or necessary. Repositioning may include moving the medical device, such as by advancing or retracting a portion of the medical device, such as a flap of the underlay member, relative to the perforation in the tympanic membrane, rotating the medical device with respect to the perforation in the tympanic membrane with our without advancing or retracting a portion of the medical device, such as a flap of the underlay member, relative to the perforation in the tympanic membrane, or other movement of the medical device relative to the perforation in the tympanic membrane. The method 900 can be used in the repair of a perforation in a tympanic membrane of an animal, such as by allowing a period of time to pass that is sufficient for an underlay member formed of a remodelable material to remodel or otherwise become incorporated into tissue of the animal in which the method is being performed.

### Example—Medical Device for Repairing Perforation in a Tympanic Membrane

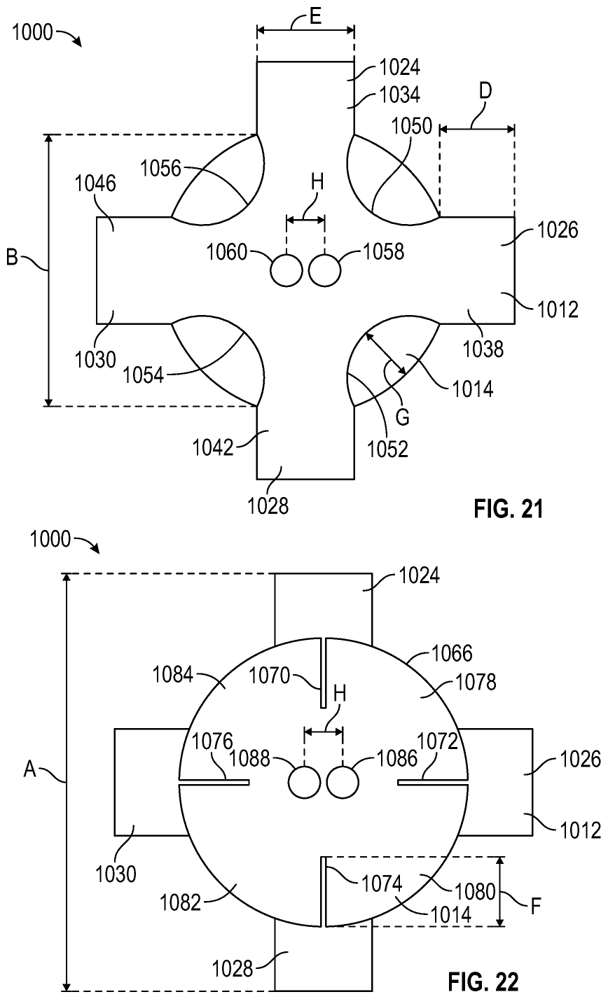

Each of FIG. 21 and FIG. 22 illustrates an example medical device 1000 according to an embodiment. The medical device 1000 includes an overlay member 1012, an underlay member 1014, and a securement member (not illustrated in FIG. 21 and FIG. 22) that secures the overlay member 1012 and the underlay member 1014 to each other. The overlay member 1012 has outwardly-extending first 1024, second 1026, third 1028, and fourth 1030 arms, each of which defines a respective arm tab 1034, 1038, 1042, 1046. The overlay member 1012 defines first 1058 and second 1060 passageways, each of which extends through the entire thickness of the overlay member 1012. The underlay member 1014 has a peripheral edge 1066 and defines multiple slits 1070, 1072, 1074, 1076 that, individually, extend from the peripheral edge 1066 toward, but not to, a geometric center of the underlay member 1014. The underlay member 1014 defines multiple flaps 1078, 1080, 1082, 1084, each of which extends between circumferentially adjacent slits 1070, 1072, 1074, 1076. The underlay member 1014 defines first 1086 and second 1088 passageways, each of which extends through the entire thickness of the underlay member 1014.

The medical device 1000 has a configuration and dimensions that make it suitable for use in the repair of a perforation in a tympanic membrane of a human being. Selected dimensions and dimension ranges of the medical device 1000 are provided in Table I and Table II below. In each Table, the letter at the beginning of each column header corresponds to the referenced dimensional indicator in one or both of FIGS. 21 and 22.

TABLE I
Selected Dimensions and Dimension Ranges of an
Example Medical Device

| A-Total | | B-Outer | | D-Tab | E- | E-Tab |
|---|---|---|---|---|---|---|
| Device | B-Outer | Diameter | D-Tab | Length | Tab | Width |
| Diameter | Diameter | Range | Length | Range | Width | Range |
| (mm) | (mm) | (mm) | (mm) | (mm) | (mm) | (mm) |
| 6.0 | 4.0 | 3.8 to 4.5 | 1.0 | 0.8 to 1.5 | 2.0 | 1.6 to 2.0 |
| 8.0 | 6.0 | 5.8 to 6.5 | 1.0 | 0.8 to 1.5 | 4.0 | 3.6 to 4.0 |

TABLE II
Selected Dimensions and Dimension Ranges of an
Example Medical Device

| | | | | | | Thread |
|---|---|---|---|---|---|---|
| | F- | | | | | Tail |
| F- | Underlay | G- | G-Wedge | | | Length |
| Underlay | Slits | Wedge | Cut-outs | H- | H-Joint | for Knot |
| Slits | Length | Cut-outs | Diameter | Joint | Width | (not |
| Length | Range | Diameter | Range | Width | Range | shown) |
| (mm) | (mm) | (mm) | (mm) | (mm) | (mm) | (mm) |
| 1.0 | 0.7 to 1.3 | 0.8 | 0.6 to 1.3 | 1.5 | 1 to 2 | 1 to 2 |

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simply to describe and illustrate examples of the invention and are not intended to limit the scope of the invention or its protection, which is to be given the full breadth of the appended claims and any and all equivalents thereof.

What is claimed is:

1. A medical device for repairing a defect in a tissue of an animal, said medical device comprising:
an overlay member comprising an overlay member body having a first overlay member surface, a second, opposing overlay member surface, and at least one arm, the overlay member body defining a first overlay member passageway extending through the overlay member body from the first overlay member surface to the second overlay member surface;
an underlay member comprising an underlay member body having a first underlay member surface and a second, opposing underlay member surface, the underlay member body defining a peripheral edge, at least one slit extending inwardly from the peripheral edge, and a first underlay member passageway extending through the underlay member body from the first underlay member surface to the second underlay member surface; and
a securement member extending through the first overlay member passageway and the first underlay member passageway.

2. The medical device according to claim 1, wherein the overlay member body defines a first curvilinear edge disposed between and connecting the first and second arms, a second curvilinear edge disposed between and connecting the second and third arms, a third curvilinear edge disposed between and connecting the third and fourth arms, and a fourth curvilinear edge disposed between and connecting the fourth and first arms.

3. The medical device according to claim 1, wherein the underlay member comprises a bioremodelable material.

4. The medical device according to claim 3, wherein the underlay member comprises a synthetic material.

5. The medical device according to claim 3, wherein the underlay member comprises a natural material.

6. The medical device according to claim 5, wherein the underlay member comprises a collagenous material.

7. The medical device according to claim 6, wherein the underlay member comprises extracellular matrix.

8. The medical device according to claim 3, wherein the overlay member comprises a bioremodelable material.

9. The medical device according to claim 3, wherein the overlay member comprises a synthetic material.

10. The medical device according to claim 9, wherein the overlay member comprises a foam.

11. The medical device according to claim 5, wherein the securement member comprises a bioremodelable material.

12. The medical device according to claim 1, wherein the underlay member and the overlay member are formed of the same material.

13. The medical device according to claim 1, wherein the overlay member defines a second overlay member passageway extending through the overlay member body from the first overlay member surface to the second overlay member surface;
wherein the underlay member defines a second underlay member passageway extending through the underlay member body from the first underlay member surface to the second underlay member surface; and
wherein the securement member extends through the second overlay member passageway and the second underlay member passageway.

14. A medical device for repairing a defect in a tissue of an animal, said medical device comprising:
an overlay member comprising an overlay member body having a first overlay member surface, a second, opposing overlay member surface, a first arm, a second arm, a third arm, and a fourth arm, the overlay member body defining a first curvilinear edge disposed between and connecting the first and second arms, a second curvilinear edge disposed between and connecting the second and third arms, a third curvilinear edge disposed between and connecting the third and fourth arms, and a fourth curvilinear edge disposed between and connecting the fourth and first arms, a first overlay member passageway extending through the overlay member body from the first overlay member surface to the second overlay member surface, and a second overlay member passageway extending through the overlay member body from the first overlay member surface to the second overlay member surface;
an underlay member comprising an underlay member body having a first underlay member surface and a second, opposing underlay member surface, the underlay member body defining a peripheral edge, a first slit extending inwardly from the peripheral edge, a second slit extending inwardly from the peripheral edge, a third slit extending inwardly from the peripheral edge, a fourth slit extending inwardly from the peripheral edge, a first underlay member passageway extending through the underlay member body from the first underlay member surface to the second underlay member surface, and a second underlay member passageway extending through the underlay member body from the first underlay member surface to the second underlay member surface; and
a securement member extending through the first overlay member passageway, the first underlay member passageway, the second underlay member passageway, and the second overlay member passageway.

15. The medical device according to claim 14, wherein the underlay member comprises a natural material.

16. The medical device according to claim 15, wherein the underlay member comprises a bioremodelable material.

17. The medical device according to claim 16, wherein the underlay member comprises an extracellular matrix material.

18. The medical device according to claim 16, wherein the underlay member comprises small intestine submucosa.

19. The medical device according to claim 16, wherein the overlay member comprises a synthetic material.

20. A medical device for repairing a defect in a tissue of an animal, said medical device comprising:
a cross-shaped overlay member formed of a synthetic material, the overlay member comprising an overlay member body having a first overlay member surface, and a second, opposing overlay member surface, the overlay member body defining a first overlay member passageway extending through the overlay member body from the first overlay member surface to the second overlay member surface;
an underlay member formed of a bioremodelable material, the underlay member comprising an underlay member body having a first underlay member surface and a second, opposing underlay member surface, the underlay member body defining a peripheral edge, a slit extending inwardly from the peripheral edge, and a first underlay member passageway extending through the underlay member body from the first underlay member surface to the second underlay member surface; and
a securement member extending through the first overlay member passageway and the first underlay member passageway.
